(19) United States
(12) Patent Application Publication
(10) Pub. No.: US 2009/0030513 A1
(43) Pub. Date: Jan. 29, 2009

(54) Multifocal Phakic Intraocular Lens

(76) Inventors: Igor G. Valyunin, Hoboken, NJ (US); Alexander P. Hatsis, Massapequa, NY (US)

Correspondence Address: FULBRIGHT & JAWORSKI, LLP, 1301 MCKINNEY, SUITE 5100, HOUSTON, TX, 77010-3095, US

(21) Appl. No.: 11/829,615

(22) Filed: Jul. 27, 2007

(51) Int. Cl. A61F 2/16

(52) U.S. Cl. 623/6.14; 623/6.27; 623/6.28; 623/6.43

(57) ABSTRACT

The invention relates to a multifocal phakic intraocular lens having three focal regions having varying radii of curvature. When properly structured and placed, the lenses of the invention provide near, far and intermediate vision. The lens may also comprise a fenestration which permits flow of aqueous through the fenestration to provide nutrition to the anterior lens epithelium as well as the corneal endothelium.

### CROSS-REFERENCE TO RELATED APPLICATIONS

Not applicable.

### STATEMENT REGARDING FEDERALLY SPONSORED RESEARCH OR DEVELOPMENT

Not applicable.

### REFERENCE TO A COMPACT DISK APPENDIX

Not applicable.

### TECHNICAL FIELD

The invention relates to multifocal intraocular lenses. More particularly the invention relates to multifocal intraocular lenses for use in a phakic eye, that is an eye where the actual lens is still in place or for use in a pseudophakic eye, that is an eye where the actual lens has been removed or replaced by a synthetic substitute.

### BACKGROUND OF THE INVENTION

Over time as a human eye ages it gradually loses the ability to accommodate. The natural aspheric lens of the eye typically remains clear and otherwise functionally unchanged for seeing in the distance by directing all of the entering rays of light to a single point on the retina. The peripheral lens area has a different anterior radius of curvature than the central lens area. This is known as an aspheric lens profile. The asphericity of the anterior surface of the natural lens directs the peripheral rays to the same focus point as the central rays even though the angle of incidence is different for the two regions. As we age, the natural loss of accommodation however, leaves people with an inability to focus at near objects because of, among other reasons, the aspheric nature of the lens. The mainstay of treatment for this condition is reading glasses or bifocals. Another approach is to wear bifocal contact lenses or contact lenses with one eye able to see near and the other eye at distance. This is called monovision. LASIK corneal surgery can produce this same type of outcome irreversibly with one eye corrected for distance and the other for near. Recently, several surgical procedures including multifocal LASIK and pseudophakic implants have been used to correct presbyopia. These procedures are irreversible and involve the permanent removal of healthy tissue from either the cornea or the lens of the eye to provide an optical system that can focus light at distance and at near simultaneously. These procedures are irreversible which is a major concern if the patient is not satisfied with the outcome or quality of vision. Thus there remains a need for additional options to correct presbyopia.

One method mentioned above is the implantation of multifocal pseudophakic intraocular lenses such as those disclosed in U.S. Pat. No. 6,790,232. These lenses have zones of different optical powers which provide the user with near, far and intermediate vision. One drawback of these types of lenses is that there is a dramatic shift in power between zones without transition which can result in a reduced visual clarity and an overall reduction in contrast sensitivity. For example, the transition regions can cause visual aberrations such as glare and halos. It is desirable therefore to design a reversible multifocal lens which preserves excellent vision while correcting presbyopia.

### BRIEF SUMMARY OF THE INVENTION

The invention relates to a phakic intraocular lens (IOL) with multifocalty to correct presbyopia. The IOL comprises a lens body with an optic having one surface, typically the anterior surface, with at least three focal regions, the first and third having about the same radius of curvature and the second having a different radius of curvature. The opposite surface, typically the posterior surface, can but not necessarily have a constant radius of curvature along its entire length. The lenses of the invention are sized and shaped for placement in either the anterior or posterior chamber of the eye. When properly structured and placed, the first region provides distance vision, the second region provides near vision and the third provides intermediate vision.

The lens is structured in a way that the focal points for each region are not the same, but are in close proximity, so that the patient does not perceive significant difference in vision, and is satisfied with the vision at all distances.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 is a cross-section of an intraocular lenses of the invention;

FIG. 2 is a top view of an intraocular lens of the invention.

FIG. 3 is a schematic showing the relative location of the focal points for the three regions of the intraocular lens.

### DETAILED DESCRIPTION OF THE INVENTION

The invention is a multifocal intraocular lens having a novel coordinated blended radii of varying curvature across specific zones of the lens optic. The zones provide a unique coordinated blend of focus points for near, intermediate and distance vision. These zones can be on either the anterior or posterior surface of the lens with the anterior surface being typical. The opposite surface, typically the posterior surface, may have a single radius of curvature.

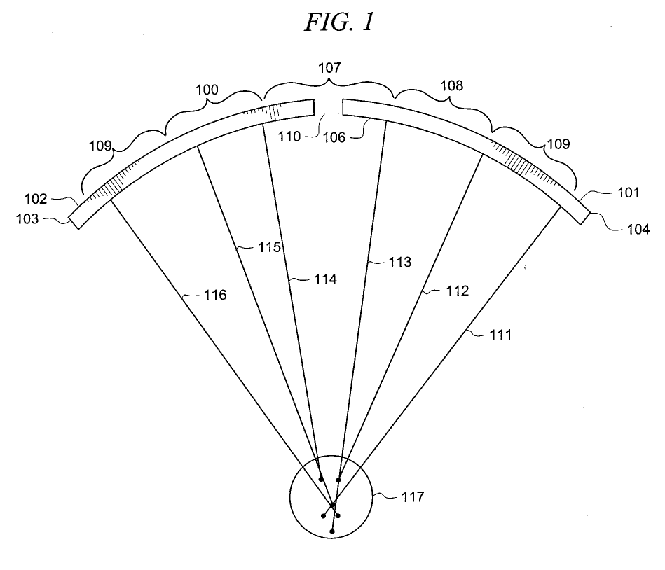

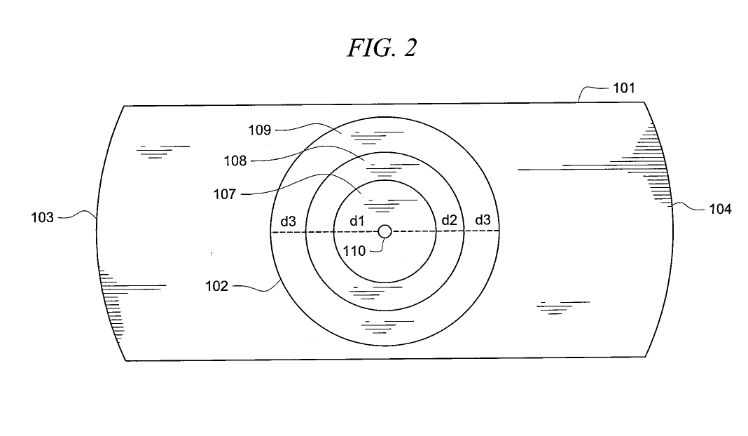

Referring to FIGS. 1 and 2, the phakic intraocular lens 101 comprises a lens body, 102 and one or more haptic bodies, 103, 104. The lens body 102 comprises an anterior surface 105 and a posterior surface 106. The anterior surface has a radius of curvature which varies by utilizing a novel coordinated system of optics creating three blended regions which provide different optical properties. The posterior surface, 106 of the lens body 102 can but not necessarily have an unchanged radius of curvature across the entire lens optic. The anterior first region 107 is typically located in the center of the lens body 102. It has a radius of curvature r₁ which provides a focus at optical point fp₁. The actual radius of curvature will vary depending on such factors as the patient and the optical properties of the material used to fabricate the lens. For example, using a material with a refraction index of approximately 1.43, the first region will have a diameter d₁ of from about 2.0 mm to 3.0 mm along the anterior surface 106 of the lens body 102 and will have a radius of curvature r₁ of about 6.7 mm to 10.0 mm. In one embodiment, the first region provides the user with distance vision.

The second region 108 has a radius of curvature r₂ which is different from the radius of curvature r₁. Again, r₂ will depend upon several factors such as the patient's needs and the refraction index of the material used to form the lens. The second region will have a radius of curvature r₂ of from about 6.0 mm to about 7.0 mm for improved near vision by providing a second optical focus point at fp₂. The second region is usually concentric with the first region. In one embodiment, the second region will have an inner diameter d₂ from about 2.0 mm to about 3.0 mm and an outer diameter d₃ of from about 4.0 mm to about 5.0 mm.

The third region 109 of the lens body 102 will have an anterior radius of curvature r₃ which is about equal to the first region (r₁≈r₃). R₃ however is designed to provide focus at optical point fp₃, therefore fp₁≠fp₃. In one embodiment r₃ will range from about 6.7 mm to about 10.0 mm. In this embodiment, the third region provides the user with simultaneous intermediate and distance vision. The third region is usually concentric with the second region and in the embodiment, has an inner diameter of d₄ of from about 4.0 mm to about 5.0 mm and has an outer diameter d₅ of from about 6.0 mm to about 7.0 mm. An important feature of this optic design is that almost equal radii of curvature for the first and third regions (r₁≈r₃) results in different focal points (fp₁≠fp₃) providing simultaneous coordinated distance and intermediate vision with a similar optic radii in a single lens optic.

While the figures and discussion above discloses an anterior surface having varied radii of curvature, one skilled in the art will appreciate that either region with varied radii of curvatures can be located on the posterior surface. In either case, the opposite face may have a single radii of curvature addressing a specific optic need such as myopia or hyperopia.

The phakic intraocular lens is centered in the posterior chamber of the eye by the following newly described tongue and grove type proper fit mechanism. In at least one embodiment, the phakic intraocular lens of the invention comprises a haptic edge arc radius of curvature equal to the specific, directly measured arc radius of curvature of the ciliary sulcus of the eye. The lens haptic edge arc curvature coupled with the overall diameter of the lens places the lens haptic edge over what we describe as the newly identified anatomic landmark we call the ciliary platform. This newly identified anatomic landmark is the area in the posterior chamber between the anterior broader of the ciliary sulcus at the posterior iris root and the posterior edges of the ciliary processes. It has been observed that the ciliary sulcus of the eye is not perfectly spherical and that the axis of its widest diameter can be directly measured. A lens of appropriate diameter and edge arc curvature is placed in this widest axis. Lens centration is assured by a predetermined fit at the widest ciliary sulcus point. The lens remains positioned by a predetermined fit or tongue and grove mechanism and is unable to rotate to a narrower axis position while floating above and not requiring to be in contact with any ciliary structures in the posterior chamber. It remains constantly anterior to the surface of the crystalline lens capsule by at least two features: (1) The invention is made of a high refractive index, hydrophobic material which by nature of its physical propriety repels the surrounding aqueous. The physical layer of aqueous between the implant lens and capsule separates it from the anterior capsule. (2) The flow of aqueous is generated by the ciliary body epithelium posterior to the implant and flows anterior being caught between the implant and anterior lens capsule. Since the implant lens is not fixed in the posterior chamber, it is propelled anterior and away from the natural lens capsule by the aqueous flow passing under the implant and out of the fenestrated aperture which will be described below. As the natural crystalline lens grows over time, the space between the implant and anterior capsule will remain relatively constant due to these features.

In at least one embodiment, the phakic intraocular lens of the invention also comprise as fenestrated aperture 110, situated in the center of the lens body 102 and extending from the anterior surface to the posterior surface of the lens body. The fenestrated aperture, which is referred to as a fenestration, allows for the aqueous fluid flow between the posterior and the anterior chambers of the eye. The fenestration eliminates the need for multiple permanent surgical iridotomies which divert the flow of aqueous peripherally and away from its natural central course. The fenestration additionally maintains a natural centripetal flow of aqueous to nourish the anterior surface of the natural crystalline lens of the eye. The fenestration also allows for a natural centripetal physiologic flow of aqueous across the endothelium appears to be beneficial for nourishment and metabolism of the eye.

The phakic intraocular lenses of the invention are typically composed from a biocompatible, flexible material. Material useful in the production of the invention includes silicone, polymer, polymethaciylude, polyhydoxy, ethylene methacrylates, collagenlacylic blends, polyhydroxyl methacrylates, and other material which may be hydrophobic, hydrophilic or gas permeable and have different refractive indices. For example, materials used to prepare intraocular lenses typically have refractive indices of from about 1.41 to about 1.52.

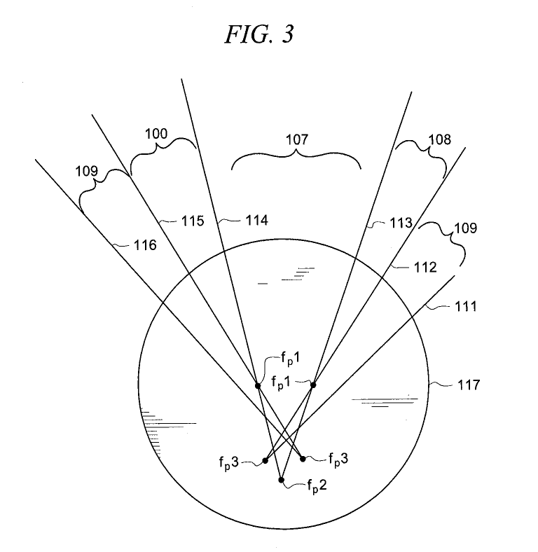

As illustrated by lines 111, 112, 113, 114, 115, and 116 each of the three regions focus light to a series of focal area located in the focal area 117. FIG. 3 is an enlargement of focal line 117 illustrating the different sets of focal points which can be achieved by practice of the invention.

As shown in FIG. 3, in one embodiment, the focal points of each region of the lenses will not be identical, but will be in sufficient proximity to each other such that the user's vision at all three distances is not noticeably affected. For example, the focal point (fp₁) for the first region 107 is slightly anterior to the focal points for the second 108 regions (fp₂) and third region (fp₃). As also shown in FIG. 3, the focal points from the third region 109 (fp₃) may be two separate focal points situated between the first fp₁, and second focal point fp₂. Again, because of the proximity of the focal points to each other, the user does not detect any or significant vision differences between the lenses of the invention and standard multifocal phakic IOLs, with respect to near, far and intermediate vision.

The intraocular lenses of the invention are employed as follows. First, the visual needs of the patient are determined including the corrections needed to provide near, far and intermediate vision. From these measurements, the required radii of curvature and relative diameters are determined. An appropriate intraocular lens is then selected from an assortment of prefabricated lenses or individually fabricated to meet the needs of the patient. The lenses are fabricated using standard manufacturing techniques using the materials recited above.

After the lens is selected or fabricated, it is then inserted into the posterior chamber of the eye using standard surgical techniques.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A phakic intraocular lens comprising:
a single unitary lens body sized and shaped for placement in the eye having at least one surface having multiple optical regions with varying powers and differing focal points to provide distance, near and intermediate vision utilizing a coordinated optical system.

2. The lens of claim 1 sized and shaped for placement in the eye; a first optical region having a first optical power and a first radius of curvature, a second region with a second optical power and a second radius of curvature different from said first optical power and said first radius of curvature and a third optical region having a radius of curvature about equal to said first radius of curvature

3. The lens of claim 1 further comprising a fenestration in the center of the lens.

4. The lens of claim 3 wherein said fenestration permits a natural physiologic centripetal flow of fluid between the anterior and posterior chambers of the eye.

5. The lens of claim 1 wherein said lens is shaped and sized for placement in the anterior chamber of the eye.

6. The lens of claim 1 where said lens is sized and shaped for placement in the posterior chamber of the eye.

7. A phakic intraocular lens comprising a unitary spherical lens body shaped and sized for placement in the eye; said lens body having a first region in the center of said lens body and having a first anterior radius of curvature; a second region concentric with the first region and having a second radius of curvature different from the first radius of curvature vision; and
a third region, concentric with said first and second region said third region having a third radius of curvature equal to the first radius of curvature.

8. The intraocular lens of claim 7 wherein said first region has a radius of curvature of from about 6.7 to about 10.0 mm.

9. The intraocular lens of claim 7 wherein said second region has a radius of curvature of from about 6.0 to about 7.0 mm.

10. The intraocular lens of claim 7 when said third region has a radius of curvature of from about 6.7 to about 10.0 mm.

11. The intraocular lens of claim 7 wherein said first and third regions have radii of curvature from about 6.7 to about 10.0 mm and said second region has a radius of curvature of from about 6.0 to about 7.0 mm.

12. The intraocular lens of claim 7 further comprising a haptic body with at least one haptic edge of predetermined arc radius of curvature.

13. The intraocular lens of claim 7 wherein the diameter of the lens is determined based on the diameter of the ciliary sulcus.

14. The intraocular lens of claim 7 wherein said first region provides distance vision.

15. The intraocular lens of claim 7 wherein said second region provides near vision.

16. The intraocular lens of claim 7 wherein said third region provides coordinated distance and intermediate vision based on varied angle of incidences.

17. The intraocular lens of claim 7 wherein the focal points of said first, second and third regions are not collocated.

18. The intraocular lens of claim 7 wherein the focal points of said first, second and third regions are in close proximity of each other.

19. The intraocular lens of claim 13 wherein said haptic body has a posterior radius of curvature similar to the radius of curvature of the natural lens and do not contract the anterior capsule.

20. The intraocular lens of claim 7 wherein said first region has a diameter of from about 2.0 to about 3.0 mm.

21. The intraocular lens of claim 7 wherein said second region has a diameter of from about 2.0 to about 3.0 mm and an outer diameter of from about 4.0 to 5.0 mm.

22. The intraocular lens of claim 7 wherein said third region has an inner diameter of from about 4.0 to about 5.0 mm and outer diameter of from about 6.0 to about 7.0 mm.

23. The intraocular lens of claim 7 further comprising an aperture in the center of said lens body, said aperture permitting fluid flow between the auteur and posterior chambers of the eye.

24. The intraocular lens of claim 7 sized and shaped for placement in the anterior chamber of an eye.

25. The intraocular lens of claim 7 wherein the lens is sized and shaped for placement in the posterior chamber of the eye.
